(19) United States
(12) Patent Application Publication
(10) Pub. No.: US 2022/0370262 A1
(43) Pub. Date: Nov. 24, 2022

(54) DIAPER

(71) Applicant: Gottlieb Binder GmbH & Co. KG, Hotzgerlingen (DE)

(72) Inventor: Konstantinos POULAKIS, Hildrizhausen (DE)

(21) Appl. No.: 17/762,791

(22) PCT Filed: Aug. 19, 2020
(86) PCT No.: PCT/EP2020/073239

§ 371 Date: Mar. 23, 2022

(30) Foreign Application Priority Data
Sep. 24, 2019 (DE) 10 2019 006 683.9

(51) Int. Cl. A61F 13/49; A61F 13/505; A61F 13/78

(52) U.S. Cl. CPC A61F 13/4906; A61F 13/505; A61F 13/78; A61F 2013/530664

(57) ABSTRACT

A diaper, such as a baby diaper or incontinence diaper, consisting of at least one outer shell (10) and having an absorbent body connected to the shell (10), is characterized in that the absorbent body can be separated from the shell by means of a mechanical fastener system acting between the outer shell (10) and the absorbent body.

The invention relates to a diaper, such as a baby diaper or incontinence diaper, consisting of at least one outer shell and having an absorbent body connected to the shell.

Such diapers, as are used in the global market, are regularly designed as disposable diapers, resulting in significant amounts of waste, the environmentally friendly disposal of which has to be considered as problematic. It is true that nowadays disposable diapers are offered on the market as so-called eco-diapers in environmentally friendly variants, which have a higher proportion of biodegradable components, at least some of which are obtained from sustainable resources; however, these eco-diapers are not disposed of via organic waste, but via the residual waste usually intended for incineration in combined heat and power plants, which cannot be described as environmentally friendly.

If (reusable) cloth diapers are used, the ecological footprint is not necessarily better than that of disposable diapers according to the content of current studies, because they have to be washed for reuse.

In practice, it has already been suggested to recycle disposable diapers—the highly absorbent cellulose as the absorbent body is to be used as cat litter after separation from the other components by dissolving the glued layers and their cleaning (source: Süddeutsche.de Wirtschaft dated Jan. 5, 2019)—; this type of recycling alone may also prove to be problematic for environmental protection reasons with regard to the separate collection of used diapers and their transport to a central recycling and processing facility.

Based on this prior art, the invention therefore addresses the problem of improving the life cycle assessment of disposable diapers of the type mentioned.

A diaper having the features of claim 1 in its entirety solves this problem.

Because in accordance with the characterizing part of claim 1, the absorbent body can be separated from the shell by means of a mechanical fastener system acting between the outer shell and the absorbent body, the used absorbent body, in particular if it is made of ecologically degradable cellulose materials, can be mechanically separated from the other components, such as the outer shell of the diaper, and disposed of in the organic waste, and the outer shell, in particular if it is made of conventional decomposable plastic materials, can be returned to the recycling cycle, which in Germany is represented by the “Yellow Bag” or any other recycling within the framework of the “Green Dot”. Because the focus is on mechanical separation, which can be performed promptly and using low actuation forces, even by the end user, in a hygienically proper manner, any loosening of adhesive joints can be dispensed with, wherein the adhesives used also have to be classified as more problematic than the mechanical joint solution proposed here for reasons of environmental protection.

In an advantageous embodiment of the diaper according to the invention, provision is made for said fastener system to comprise a hook-and-loop fastener consisting of hooks and/or mushroom heads on one side and loops or fibers or further mushroom heads that can be hooked thereto in a detachable manner on the other side. A fastener system of this type and a method of producing it are known from DE 196 46 318 A1 of the proprietor. Fastener systems, in which mushroom heads on both sides of the fastener material are latched together in a re-detachable manner, are also known technically as hermaphroditic fasteners.

In particular, in an advantageous manner in the diaper according to the invention, provision is further made for the loops or fibers of the one part of the hook-and-loop fastener to be formed of at least one interlaced fabric, knitted fabric, woven fabric, gauze, fleece, nonwoven fabric or velour. Basic fabrics of this type which are made of warp and weft yarns and which have functional elements in the form of loops projecting from the front of the basic fabric, are described by way of example for a flame-retardant fastener part in DE 10 2013 022 112 A1 of the proprietor.

As an alternative to the aforementioned hook-and-loop fastener, the fastener system may comprise a film having a plurality of protruding attachment heads, the end-face head surfaces of which adhere to the outer shell and/or to the absorbent body in a detachable manner by means of adhesion. The proprietor markets the fastener system in question under its trademark “Gecko” as the so-called Gecko tape, which is perceived as extremely pleasant and comfortable for the respective wearer of such a diaper because of its thin-layer design and its flexibility in all directions. Examples of such adhesion solutions are disclosed in DE 10 2010 044 660 A1 and DE 10 2010 032 855 A1 of the proprietor.

In a further, particularly advantageous manner, provision is made for the absorbent body of the diaper to have an suction body, in particular in the form of cellulose flakes, which are accommodated in a closed, fluid-permeable, absorber shell, in particular made of fleece material, which is particularly preferably biodegradable. Said fleece material of the absorber shell can in this respect form the one part of the hook-and-loop fastener, which can be connected, in particular hooked, to the other part in the form of a hook material in a detachable manner.

In a further, advantageous manner, provision is made for the entire surface of the absorber shell facing the inside of the outer shell to be hooked to the one side having the hooks or mushroom heads of the hook-and-loop fastener in a detachable manner. Alternately, it is possible to design the absorber shell with the Gecko tape in a similar way.

In a particularly preferred embodiment of the diaper according to the invention, provision is made for the outer shell to be formed of a film-like fastener material (gecko tape), the hooks or mushroom heads of which face the absorbent body. In this way, the outer shell of the diaper can be designed using only one layer of film, which increases the wearing comfort and permits disposal with an improved eco-balance.

Preferably provision is made for a separation and singulation of the hooks or mushroom heads of the hook-and-loop fastener film as the outer shell to be stretched unidirectionally and, if necessary, bidirectionally, rendering the film material thinner; at the same time, however, the tear strength is retained, which in turn reduces the residual amount of diaper to be disposed of or recycled after removal of the absorbent body, and the thinness of the film having the separation and singulation of the fastener elements again increases the wearing comfort for the user.

The invention also relates to a method for improving the ecological footprint using a diaper according to any of the preceding claims, wherein after use of the diaper the fastener system is used to separate at least the absorbent body from the reusable or recyclable outer shell for the disposal of the absorbent body.

The diaper according to the invention will be described in greater detail by way of two exemplary embodiments. In the figures, in general view, not to scale,

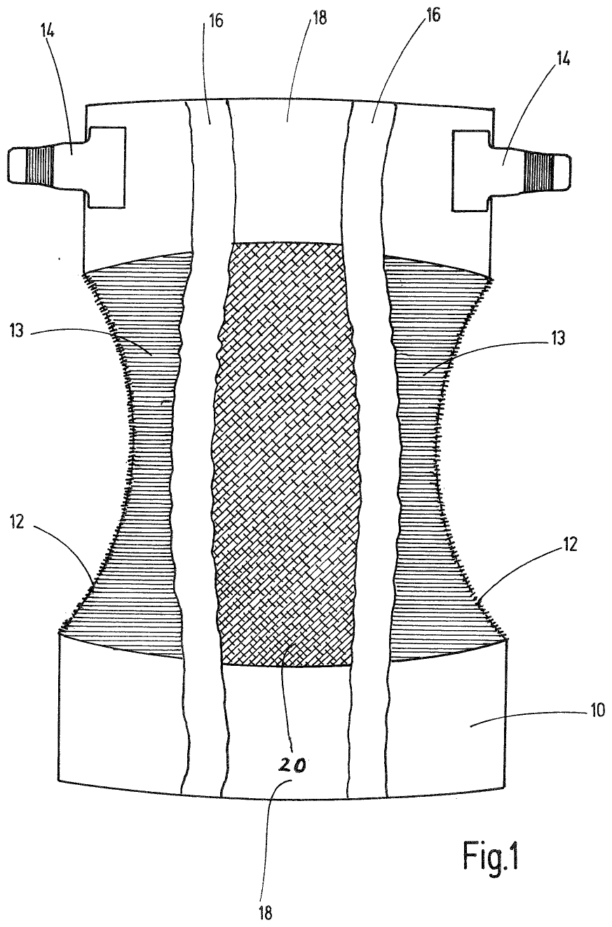

FIG. 1 shows a top view of a first exemplary embodiment of the diaper;

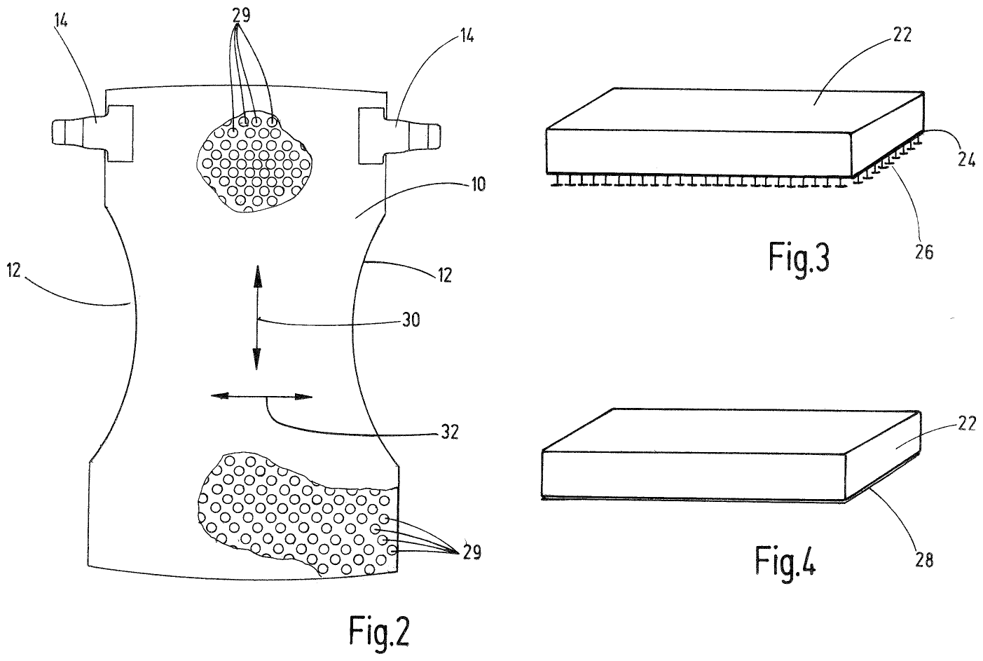

FIG. 2 shows, corresponding to FIG. 1, a representation of a second exemplary embodiment of the diaper, and

FIGS. 3 and 4 show two fastener system parts different from each other for an absorbent body as can be used for a diaper according to FIGS. 1 and 2.

FIG. 1 shows a flat projection of the outer shell 10 of a disposable diaper in the form of a baby diaper or an incontinence diaper for adults. The outer shell 10 is tapered along its two longitudinal edges 12 opposing each other, such that leg openings for a diaper user are formed in that way. The two longitudinal sections 13 forming a kind of cuff, are formed of an elastic material common to diapers, which increases the wearing comfort of the user. At the upper end, as viewed in the direction of FIG. 1, of the outer shell 10, two fasteners 14 are attached in a customary manner, by means of which the diaper can be secured to a user, and closed after use, rolled up. For this purpose, the two fasteners have, in the direction of their two free ends, an adhesive or hook-and-loop fastener parts (both not shown), by which the fasteners 14 on the front of the diaper in a so-called target area permit a permanently attachment of the diaper.

Along the two longitudinal edges 12 two cover strips 16 extend continuously, as viewed in the direction of FIG. 1, to the right and left of the center of the shell 10 and from top to bottom, wherein a part 20 of a hook-and-loop fastener is arranged between said two cover strips 16, leaving two end sections 18 blank. The cover strips 16 are firmly connected to the shell 10 in the direction of the two longitudinal edges 12 and can be lifted off in a pocket-like manner in the direction of their two adjacent edge sections to be able to hold an absorbent body 22 in this way, overlapping at the edges and covering in this respect. In this exemplary embodiment, the part 20 is made of a loop material, which is inserted as a continuous strip under the two free ends, facing each other, of the cover strips 16 and is firmly connected, for instance firmly glued, to the outer shell 10. However, instead of a stand-alone loop material, a fiber material of the outer shell 10 may be directly provided for the hooking process still to be described.

A suitable absorber material is shown in FIG. 3 by way of example in form of the absorbent body 22, which has a fluid-permeable gauze shell enclosing the cellulose flakes inside the absorbent body 22, wherein said cellulose flakes are used to absorb fluid, typically formed from the excretions of the diaper user.

On the underside of the strip-shaped or block-shaped absorbent body 22, a fastener material in the form of hooks 26, which projects downward as viewed in the direction of FIG. 3, is provided in a planar manner on a backing tape 24. When the absorbent body 22 equipped in this way, is placed onto the flat projection of the shell 10 according to FIG. 1, the hooks 26 on one side of the proposed hook-and-loop fastener interlock with the loops on the other side of the hook-and-loop fastener, and to that extent form a re-detachable mechanical fastener. In the merchantable state of sale, the diaper is delivered with the absorbent body 22 connected via the mechanical fastener system in this way and is thus ready for use. After putting on the diaper and its subsequent use, the absorbent body 22 is appropriately soiled and the mechanical fastener system consisting of hooks 26 and loops of the part 20, can be separated from the remaining outer shell 10 of the diaper. The absorbent body 22, which consists of biodegradable materials, can then be disposed separately from the outer shell 10, for instance as part of the organic waste. The outer shell 10 can then in turn be disposed in a supply circuit separated therefrom for the purpose of obtaining reusable raw materials. It would also be conceivable, for instance in clinics or care facilities, to deliver only one outer shell 10 in a package in conjunction with a plurality of absorption bodies 22, which can then be exchanged for new bodies 22 after use while retaining the individual outer shell 10 for a certain predeterminable period of use.

If the velour or fiber material of a diaper in the form of the outer shell 10 is used directly as the fastening zone, no separate part 20 of the hook-and-loop fastener in the form of the loop material is required, so that there is also no need to provide an adhesive connection between the outer shell 10 and the independent part 20 of the hook-and-loop fastener, which in turn helps to reduce manufacturing costs and promotes the environmentally friendly design of the diaper.

As explained above, the aids referred to as disposable diapers or diaper pants have the presented outer shell 10, which is regularly made of polyethylene materials and has an absorbing body. Recent developments make it possible to at least partially manufacture this polyethylene from sugar cane, which, as a renewable raw material and thus a biobased production material, can be classified as very environmentally friendly. If necessary, the diaper can also be constructed from bio-based polymer material or polypropylene.

As explained above, the absorbent body 22 consists of cellulose materials, in particular in flake form, which in modern diapers are often enriched with an additional superabsorbent and specific polymer salts to bind high amounts of liquid in the volume of the absorbent part of the absorbent body 22. In particular, under the wearing pressure, the liquid once absorbed is not released again, unlike for a sponge. On the inner or outer layer of the shell 10, the adhesive or Velcro tabs are provided as fasteners 14, by means of which the diaper can be put on and, in particular, be adjusted to the abdominal circumference of the user. They may be coated with a plastic film to prevent liquids from seeping out of the diaper. Further diaper ingredients may include Vaseline, stearyl alcohol, thin paraffins and extracts of aloe barbadensisis. Provided that natural materials are used here; such additional materials and additives do not impair the environmentally friendly recycling and reuse of the diaper according to the invention.

It is still within the scope of the invention to replace the hooks 26 of the absorbent body 22 with loops, and the loop tape as the part 20 of the outer shell 10 with a matching hook fastener material. In addition to the above-mentioned hook-loop fastener system, the hooks 26 can also be replaced in the usual manner by mushroom heads as the one fastener part, which can be connected in a detachable manner to a matching mushroom head material on the side of the outer shell 10 while forming so-called hermaphroditic fasteners. In particular, if the absorbent body 22 is to be connected directly to the fiber or fleece material of the outer shell 10, it may be advisable to use the so-called Gecko tape 28, as shown in FIG. 4 by way of example. On its underside, the aforementioned gecko tape 28 has fastener elements in the form of mushroom heads, whose geometric dimensions, produced using microreplication technology, are that small that they are barely visible to the naked eye on their backing film. The free end faces of the micro-mushroom heads allow adhesion to the outer shell 10 of the diaper predominantly by applying adhesive forces between the outer shell 10 and the gecko film 28. If the absorbent body 22 consists of a planar absorber material, the cellulose shell for the body 22 can also be omitted, if necessary, and the respective hook-and-loop fastener part provided can then also form the casing of the absorbent body directly connected to the absorbent body, wherein the respective fastener part is to be designed to be fluid-permeable by inserting a perforation. In such embodiments, it would also be conceivable to provide the backing tape 24 as shown in FIG. 3 with a corresponding fastener material, hooks or mushroom heads on both sides, such that one side of the fastener material is used to connect to the absorber material and the other free side is available for connection to the diaper outer shell 10 or the part 20.

The outer shell 10 in the modified embodiment shown in FIG. 2 essentially consists of a flat cut sheet of plastic material. On the inside facing the viewer, of the outer shell 10, a fastener material in the form of mushroom heads 29, arranged in predeterminable columns and rows relative to one another, is arranged in a continuous manner. To increase the wearing comfort and to keep the amount to be disposed of small, the outer shell 10 has been stretched unidirectionally, viewed in longitudinal direction, as arranged by the arrows 30. However, it is also possible, if necessary, to stretch it transverse to the extending direction of the double arrows 30, i.e. in accordance with the arrow illustration 32, or to stretch or elongate the film material of the outer shell 10 bidirectionally both in the longitudinal direction 30 and transversely thereto 32. Exemplary stretching methods are disclosed in U.S. Pat. No. 4,001,366 and in DE 694 21 025 025 T2.

In this embodiment, the fibrous absorber material of the absorbent body 22 is directly connected to the outer shell 10 on the inside thereof. It is also possible to use the absorbent body 22 shown in FIG. 3, with the proviso that the backing tape 24 then does not have hook fastener material 26 on its underside, but rather loop-shaped connecting bodies (not shown) as part of the hook-and-loop fastener system. Also, instead of hooks as fastening elements for loops, a mushroom head design can be used. In view of the fact that several billion disposable diapers have to be disposed of every year worldwide, this invention is of great importance in terms of evident waste prevention. The absorbent body can also be made of compostable materials.

1. A diaper, such as a baby diaper or incontinence diaper, consisting of at least one outer shell (10) and having an absorbent body (22) connected to the shell (10), characterized in that the absorbent body (22) can be separated from the shell (10) by means of a mechanical fastener system acting between the outer shell (10) and the absorbent body (22).

2. The diaper according to claim 1, characterized in that the mechanical fastener system comprises a hook-and-loop fastener consisting of hooks (26) and/or mushroom heads (29) on one side and loops (20) or fibers or further mushroom heads that can be hooked thereto in a detachable manner on the other side.

3. The diaper according to claim 1, characterized in that the loops or fibers on the other side are formed from at least one interlaced fabric, knitted fabric, woven fabric, gauze, fleece, nonwoven fabric or velour.

4. The diaper according to claim 1, characterized in that the fastener system comprises a film (28) having a plurality of protruding attachment heads, the end-face head surfaces of which adhere to the outer shell (10) and/or to the absorbent body (22) in a detachable manner by means of adhesion.

5. The diaper according to claim 1, characterized in that the absorbent body (22) has a suction body, in particular in the form of cellulose flakes, which are accommodated in a closed, fluid-permeable absorbent shell, in particular made of fleece material.

6. The diaper according to claim 1, characterized in that at least part of the surface of the absorber shell, preferably its entire surface, facing the inside of the outer shell (10), which constitutes the other side of the hook-and-loop fastener, on the one side having the hooks or mushroom heads of the hook-and-loop fastener is hooked in a detachable manner.

7. The diaper according to claim 1, characterized in that at least part of the surface of the absorber shell, preferably its entire surface, on its side facing the outer shell (10), is formed from the one side having the hooks or mushroom heads of the hook-and-loop fastener or in that this one side additionally bears the film material or surface material on its outer side.

8. The diaper according to claim 1, characterized in that the outer shell (10) is formed of a film-like hook-and-loop fastener, whose hooks (26) or mushroom heads (29) face the absorbent body (22).

9. The diaper according to claim 1, characterized in that for a separation and singulation of the hooks or mushroom heads of the adhesive fastener film as the outer shell (10), it is stretched unidirectionally, preferably bidirectionally.

10. A method for improving the ecological footprint using a diaper according to claim 1, characterized in that, after use of the diaper, at least the absorbent body (22) is separated from the reusable or recyclable outer shell (10) via the mechanically actuatable fastener system for the disposal of the absorbent body (22).
